(19) United States
(12) Patent Application Publication
(10) Pub. No.: US 2011/0015256 A1
(43) Pub. Date: Jan. 20, 2011

(54) DELIVERY OF RESTRICTION ENDONUCLEASES TO TREAT HIV, CANCER, AND OTHER MEDICAL CONDITIONS

(76) Inventor: Ihab Mamdouh Ishak Sadek, El Mina (EG)

Correspondence Address: SHELDON MAK & ANDERSON PC, 100 Corson Street, Third Floor, PASADENA, CA, 91103-3842, US

(21) Appl. No.: 12/504,279

(22) Filed: Jul. 16, 2009

(51) Int. Cl. A61K 31/7088

(52) U.S. Cl. 514/44 R

(57) ABSTRACT

A method of treating a medical condition caused by the presence of an undesired DNA sequence in a predetermined cell type of a subject by administering to the subject one or more restriction endonucleases to specifically cleave the undesired DNA sequence.

### BACKGROUND

Cells can contain undesired polynucleotide sequences, either due to abnormalities in their genetic material, which can lead to cancer, or through infection with a virus. According to the World Heath Organization, cancer causes about 13% of all human deaths. (WHO, 2006). Once diagnosed, cancer is usually treated with a combination of surgery, chemotherapy and radiotherapy. Complete removal of the cancer can sometimes be accomplished by surgery, but the propensity of cancers to invade adjacent tissue or to spread to distant sites by microscopic metastasis often limits its effectiveness. The effectiveness of chemotherapy is often limited by toxicity to other tissues in the body. Radiation can also cause damage to normal tissue.

Viruses can cause many common human diseases, including the common cold, influenza, chickenpox and cold sores. Many serious diseases such as ebola, AIDS, avian influenza and SARS are caused by viruses. A virus can have either a DNA or RNA genome. Retroviruses, which contain RNA genomes, use the reverse transcriptase enzyme to convert RNA into DNA and often integrate their DNA into the host DNA genome. An example of a retrovirus is Human Immunodeficiency Virus (HIV). Antiviral treatments include those that target different stages of the viral life cycle. However, treatment with antiviral drugs has drawbacks, including that viruses may develop drug resistance over time and become less susceptible to treatment.

Prokaryotic cells have been known to contain endonucleolytic restriction enzymes (Gingeras, 1991, Modern Microbial Genetics 301-321 (Wiley-Liss, Inc.). The restriction endonucleases recognize a specific sequence in viral DNA and subsequently cleaves both strands of the DNA into fragments, which are then degraded further by other endonucleases. Restriction endonucleases have not been found pervasively in eukaryotic cells, although restriction endonucleases have been isolated from several eukaryotic sources. (Sklar et al., 1986, J. Biol. Chem. 261: 6806-6810; Lao et al., 1986, Sci. Sin. (Series B) 29: 947-953).

One reported study (Kwoh et al., 1986, Proc. Natl. Acad. Sci. USA 83: 7713) unsuccessfully attempted to utilize restriction endonucleases expressed by eukaryotic cells to inhibit viral infection. U.S. Pat. No. 5,523,232 to Sechler further proposed treating various conditions by administering restriction endonucleases to a subject. In another study of the effect of endonuclease activity, mutant mitochondrial DNA was eliminated in cybrid cells through the transient expression of the endonuclease SmaI (Tanaka et al., Journal of Biomedical Science, 2002, 9: 534-541).

### SUMMARY

The present invention relates to treating a medical condition in a subject in which the medical condition is caused by the presence of an undesired DNA sequence in a predetermined cell type. The method treats the subject with the medical condition by administering to the subject a one or more restriction endonucleases to specifically cleave the undesired polynucleotide.

The present method provides for the treatment of a medical condition characterized by the presence of an undesired DNA sequence in a predetermined cell type. Such conditions include viral infections such as HIV, bacterial infections, cancer, and genetic disorders involving the accumulation of undesired proteins within the cells of a subject. The method comprises the steps of (a) providing a viral vector comprising a restriction endonuclease or one or more polynucleotides coding for the restriction endonuclease; and administering to a subject an amount of the vector capable of treating the medical condition, wherein the restriction endonuclease specifically cleaves the undesired DNA sequence.

The vector preferably is targeted to cell type in which the endonuclease is needed, and also preferably delivers the restriction endonuclease protein directly. Such vectors can be, for example, a virus or viral shell derived from HCMV or AAV, to which the endonuclease may be fused. The endonuclease can alternatively be fused to a protein transduction domain transporter peptide in order to effect entry of the endonuclease into a cell. Alternatively, the vector can deliver a polynucleotide able to be translated within a host cell in order to create a restriction endonuclease within the host cell. Such polynucleotides can be delivered as plasmids, such as with a liposome vector, or in a viral vector. The viral vector can be, e.g., a replication-defective HSV vector, an attenuated HSV vector, a baculovirus, or an alpha-virus. Vectors are preferably formulated with a pharmaceutically acceptable excipient prior to administration.

### DESCRIPTION

### Definitions

As used herein, the following terms and variations thereof have the meanings given below, unless a different meaning is clearly intended by the context in which such term is used.

The term “cancer” refers to a disease or disorder that is characterized by unregulated cell growth. Examples of cancer include, but are not limited to, carcinoma, lymphoma, blastoma and sarcoma. Examples of specific cancers include, but are not limited to, lung cancer, colon cancer, breast cancer, testicular cancer, stomach cancer, pancreatic cancer, ovarian cancer, liver cancer, bladder cancer, colorectal cancer, and prostate cancer. Additional cancers are well known to those of skill in the art.

“DNA sequence” refers to a chain of deoxyribonucleotides, i.e. oligonucleotides or polynucleotides, in either single- or double-stranded form.

“Expression” of a nucleotide sequence refers to the transcription of the sequence and its subsequent translation into a polypeptide.

“Genetic disorder” refers to a medical condition caused by abnormalities in a gene of a subject.

The term “mammal” is defined as an individual belonging to the class Mammalia and includes, without limitation, humans, domestic and farm animals, and zoo, sports, and pet animals, such as sheep, dogs, horses, cats and cows.

“Medical condition,” “disease,” and “disorder” refer to an abnormal condition of an organism that impairs one or more bodily functions and/or causes discomfort, generally associated with specific symptoms and/or other indications. The medical conditions treated by the present method are due to the presence of an undesired polynucleotide in a host cell.

As used herein, the terms “nucleic acid,” “polynucleotide” and “nucleotide” refer to molecules consisting of or comprising one or more nucleic acids, whether such nucleic acids are composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, or combinations of such linkages. The terms nucleic acid, polynucleotide and nucleotide also specifically include molecules consisting or comprised of nucleic acids composed of bases other than the five biologically occurring bases (adenine, guanine, thymine, cytosine and uracil).

“Nucleotide sequence” refers to a chain of deoxyribonucleotides or ribonucleotides, i.e. oligonucleotides or polynucleotides, in either single- or double-stranded form.

The term “operably linked” refers to functionally related nucleic acid sequences. When a promoter controls and/or enhances the transcription of a nucleotide sequence, for example, it is said to be operably linked to the nucleotide sequence.

“Promoter” refers to a nucleotide sequence or sequences, usually comprising a transcription factor binding site, which directs and/or enhances transcription of another nucleotide sequence.

A “replication defective” viruses and viral vectors are those which can transmit a polynucleotide or protein into a host cell in need of treatment with the present methods but which are unable to replicate independently in the host cell and produce progeny viruses. In some cases replication defective viruses can be replicated in a host cell with the aid of a helper virus. Replication defective virus particles (virions) generally contain all the viral structural components of the virus and can attach, penetrate, and release their nucleic acid within the host cell, but a mutation generally has destroyed an essential viral function such that new virions will not be made unless the missing function is otherwise provided, such as by a helper virus. Replication defective viruses, such as those derived from HSV-2 and AAV, are known to those of skill in the art.

“Retroviruses” are viruses having an RNA genome. Retroviruses which can be treated according to the present invention are those that replicate in a host cell by producing DNA from an RNA genome, such as with the enzyme reverse transcriptase.

“Restriction endonuclease” or “restriction enzyme” refers to an enzyme that cuts double-stranded or single stranded DNA at specific recognition nucleotide sequences, or restriction sites. Restriction endonucleases can be prokaryotic or eukaryotic in origin. Restriction endonucleases can be naturally occurring restriction endonucleases, modified naturally occurring restriction endonucleases, artificial restriction endonucleases, or a combination of the foregoing.

“Subject” refers to an animal, preferably a mammal, and more preferably a human, in need of treatment and/or treated according to the methods described herein.

As used herein, “treatment” is a clinical intervention made in response to a disease, disorder or physiological condition caused by the presence of undesired polynucleotides manifested by a patient or to be prevented in a patient. The aim of treatment includes the alleviation and/or prevention of symptoms, as well as slowing, stopping or reversing the progression of a disease, disorder, or condition. “Treatment” refers both to therapeutic treatment as well as to prophylactic or preventative measures. Those in need of treatment include those already affected by a disease or disorder or an undesired physiological condition as well as those in which the disease or disorder or undesired physiological condition is to be prevented. “Treatment” need not completely eliminate a disease, nor need it completely prevent a subject from becoming afflicted with a disease or disorder.

“Tumor,” as used herein, refers to all neoplastic cell growth and proliferation, whether malignant or benign, and all pre-cancerous and cancerous cells and tissues.

A “vector” is a polynucleotide, lipid, protein, and/or a virus or other collection of molecules, which is capable of transporting a protein such as a restriction endonuclease and/or a nucleotide sequence into a cell. Vectors can be, for example, plasmids, viruses, cosmids or phage. The vectors used in the present methods are preferably viral vectors, and more preferably are replication defective viral vectors. Viral vectors are derived from viruses but contain an exogenous protein or polynucleotide in addition to or in place of the components normally comprising the virus. An “expression vector” is a vector that is capable of directing expression of a protein encoded by one or more genes carried by the vector when it is present in the appropriate environment.

As used herein, the term “comprise” and variations of the term, such as “comprising” and “comprises,” are not intended to exclude other additives, components, integers or steps. The terms “a,” “an,” “the.” and similar referents used herein are to be construed to cover both the singular and the plural unless their usage in context indicates otherwise.

### Restriction Enzymes

It is contemplated that unmodified prokaryotic or eukaryotic restriction endonucleases can be used in the methods of the invention. A number of restriction endonucleases are known to those of skill in the art. A partial list of such endonucleases is provided in Table 1 below.

TABLE 1
Restriction Endonucleases

| | Recognition | |
|---|---|---|
| Enzyme | Sequence | Cut |
| EcoRI | 5′GAATTC | 5′---G AATTC---3′ |
| | 3′CTTAAG | 3′---CTTAA G---5′ |
| EcoRII | 5′CCWGG | 5′--- CCWGG---3′ |
| | 3′GGWCC | 3′---GGWCC ---5′ |
| BamHI | 5′GGATCC | 5′---G GATCC---3′ |
| | 3′CCTAGG | 3′---CCTAG G---5′ |
| HindIII | 5′AAGCTT | 5′---A AGCTT---3′ |
| | 3′TTCGAA | 3′---TTCGA A---5′ |
| TaqI | 5′TCGA | 5′---T CGA---3′ |
| | 3′AGCT | 3′---AGC T---5′ |
| NotI | 5′GCGGCCGC | 5′---GC GGCCGC---3′ |
| | 3′CGCCGGCG | 3′---CGCCGG CG---5′ |
| HinfI | 5′GANTC | 5′---G ANTC---3′ |
| | 3′CTNAG | 3′---CTNA G---5′ |
| Sau3A | 5′GATC | 5′--- GATC---3′ |
| | 3′CTAG | 3′---CTAG ---5′ |
| PovII | 5′CAGCTG | 5′---CAG CTG---3′ |
| | 3′GTCGAC | 3′---GTC GAC---5′ |
| SmaI | 5′CCCGGG | 5′---CCC GGG---3′ |
| | 3′GGGCCC | 3′---GGG CCC---5′ |
| HaeIII | 5′GGCC | 5′---GG CC---3′ |
| | 3′CCGG | 3′---CC GG---5′ |
| HgaI | 5′GACGC | 5′---NN NN---3′ |
| | 3′CTGCG | 3′---NN NN---5′ |
| AluI | 5′AGCT | 5′---AG CT---3′ |
| | 3′TCGA | 3′---TC GA---5′ |
| EcoRV | 5′GATATC | 5′---GAT ATC---3′ |
| | 3′CTATAG | 3′---CTA TAG---5′ |
| KpnI | 5′GGTACC | 5′---GGTAC C---3′ |
| | 3′CCATGG | 3′---C CATGG---5′ |
| PstI | 5′CTGCAG | 5′---CTGCA G---3′ |
| | 3′GACGTC | 3′---G ACGTC---5′ |
| SacI | 5′GAGCTC | 5′---GAGCT C---3′ |
| | 3′CTCGAG | 3′---C TCGAG---5′ |
| SalI | 5′GTCGAC | 5′---G TCGAC---3′ |
| | 3′CAGCTG | 3′---CAGCT G---5′ |
| ScaI | 5′AGTACT | 5′---AGT ACT---3′ |
| | 3′TCATGA | 3′---TCA TGA---5′ |
| SphI | 5′GCATGC | 5′---G CATGC---3′ |
| | 3′CGTACG | 3′---CGTAC G---5′ |
| StuI | 5′AGGCCT | 5′---AGG CCT---3′ |
| | 3′TCCGGA | 3′---TCC GGA---5′ |
| XbaI | 5′TCTAGA | 5′---T CTAGA---3′ |
| | 3′AGATCT | 3′---AGATC T---5′ |

Table 1 Notes:
N = C or G or T or A; W = A or T

Patterns of restriction endonuclease cleavage in an undesired polynucleotide can be determined readily by computerized analysis once the nucleic acid sequence of the undesired polynucleotide has been determined. Thus, known restriction endonucleases that cut a polynucleotide in specific regions can be identified as candidates for use in the present methods.

In addition, restriction endonucleases used in the methods of the invention can also be modified forms of naturally occurring restriction endonucleases. Such modified restriction endonucleases can be produced by a variety of well-known techniques such as, for example, recombinant DNA techniques, conjugation to other moieties and carriers, and chemical modification. For example, it is possible to manipulate known restriction endonucleases to recognize different DNA sequences through random mutagenesis or directed evolution (Lanio et al., 1998, J. Mol. Bio. 283: 59-69). Restriction endonucleases used in the methods of the invention include molecules that both preserve the cleavage activity of the natural enzyme as well as those that add additional structural features that provide properties desirable for removing undesired DNA from a target cell.

Restriction endonucleases can also be created through random mutagenesis, based on an unwanted polynucleotide sequence which is to be cleaved. For example, mutagenized enzymes can be assayed in order to identify those with a desired nuclease activity, i.e. with a predetermined specificity for cleaving a nucleotide sequence in an unwanted strand of DNA. Techniques used in the design and selection of DNA-binding proteins or individual domains capable of novel sequence recognition make it possible to engineer restriction endonucleases to target specific DNA sequences (Segal and Barbas, 2000, Current Opinion in Chem. Biol. 4: 34-39).

In this way, restriction endonucleases can be custom made for removal of a particular undesired sequence of DNA present in a subject. In order to identify or design a custom restriction endonuclease that recognizes an undesired polynucleotide, a sample of the affected cells can be obtained by, for example, a biopsy of tumor cells. The DNA can then be extracted from the tumor cells and sequenced in order to obtain the sequence of an undesired polynucleotide to be excised.

A fragment of an enzyme that provides sequence recognition and endonucleolytic activities can also be used as the restriction endonuclease of the present methods, alone or combined with additional structural elements. For example, the active portion of a restriction enzyme can be combined with other substituents and/or moieties by means well known in the art to provide such additional functions as decreased immunological reactivity, receptor or antigen binding or other targeting moieties, and delayed release, among others.

Certain types of restriction endonucleases cleave substrate DNA outside their recognition sequences. Biochemical and structural studies have shown that these endonucleases consist of separate DNA recognition and cleavage domains. This uncoupling of substrate specificity and cleavage activity further allows for the creation of chimeric endonucleases of novel substrate specificity by fusing DNA cleavage domains to DNA recognition proteins or oligonucleotides. (Chan et al., 2007, Nucleic Acids Research 35: 6238-6248). Such chimeric restriction endonucleases can be used in the methods of the present invention.

The restriction endonucleases expressed in the present method are preferably selected to specifically cleave a sequence in an unwanted strand of DNA and not cleave sequences present in host DNA. In order to select an appropriate sequence in the unwanted strand of DNA to cleave, the sequence of the unwanted strand can be compared to the known sequences of the host cell in which the endonuclease is to be expressed, and a sequence in the unwanted DNA strand that is not present in the known host DNA can be selected. Preferably, the entire genome of a host cell is compared with the sequence of unwanted DNA in order to select an appropriate sequence to cleave. Once an affected cell is treated with a restriction endonuclease and the undesired polynucleotide excised according to the present methods, the ends of the host DNA are also preferably ligated together, resulting in a normal cellular DNA sequence.

Restriction endonucleases used in the methods of the invention can also be modified to alter structural features that affect their ability to enter a cell nucleus. For instance, restriction endonucleases within a particular range of molecular weights may be preferred for this purpose in the invention. Natural or engineered enzymes in the range of 60,000 daltons or above generally will not diffuse into the nucleus and therefore are preferred for applications in which the sequence to be cleaved by the restriction endonuclease is outside the nuclease of a cell, such as a DNA sequence of an intracellular bacteria such as mycobacterium tuberculosis. On the other hand, if it is desired that DNA within a cell nucleus be targeted, restriction endonucleases below 60,000 daltons are preferred. Active transport of restriction endonucleases into the nucleus can be achieved by adding a nuclear targeting sequences into the amino acid sequence of the restriction endonuclease. Amino acid targeting sequences, such as those of various viral proteins, are known to the art.

### Vectors and Vector Delivery

The restriction endonuclease of the present invention can be delivered as a protein to a predetermined cell type, or as a polynucleotide encoding the restriction endonuclease followed by expression of the polynucleotide. A vector as used in the present invention preferably will not adversely affect the host cell or produce viral proteins, and so will be able to evade the cell mediated immunity of the host. Additionally, the vector is preferably a replication defective viral vector.

Viral vectors are preferred for delivering restriction endonucleases to host cells in need thereof. Viral vectors can be used which transmit polynucleotides or proteins into a number of different cell types, such as AAV vectors. Many viral vectors however have the advantage of cell type specificity, i.e. being able to deliver restriction endonucleases to specific cell types which are implicated in a particular medical condition.

Vectors for treating HIV preferably target the cells that the HIV virus infects, i.e. CD4+ cells. Preferably, viral vectors for the treatment of HIV incorporate binding domains from HIV for the CD4+. In one embodiment, the vector can be a lentiviral vector. To obtain a replication defective lentiviral vector, several plasmids can be transfected into a packaging cell line, commonly HEK 293. One or more plasmids, generally referred to as packaging plasmids, encode the virion proteins, such as the capsid and the reverse transcriptase, while another plasmid contains the endonuclease sequence to be delivered by the vector. The plasmid carrying the endonuclease is transcribed to produce a single-stranded RNA viral genome marked by the presence of the ψ (psi) sequence, which is used to package the genome into the virion. For safety reasons lentiviral vectors should not carry the genes required for their replication, and/or such genes should contain insert sequences so that the virus cannot replicate inside a host cell. [Kalpana, “Retroviral Vectors for Liver-directed Gene Therapy,” Seminar in Liver Disease, 19: 27-37 (1999); Amado, et al., “Lentiviral Vectors—the Promise of Gene Therapy Within Reach?” Science, 285:674-76 (1999)]. Targeting efficient restriction endonuclease delivery vehicles to the desired cell types with specificity greatly enhances the therapeutic potential of the endonuclease and alleviates concerns of off-target effects in vivo. For example, herpes simplex viruses (HSVs, such as HSV-1 and HSV-2) target neurons, and hepatitis viruses target liver cells. Vectors can be more specifically targeted by being adapted to bind to a specific target cell protein on a predetermined cell type in order to specifically deliver such vectors to a cell in need thereof. Other viral vectors known to those of skill in the art, such as vectors derived from HPV [Eisenberger, et al., “A Human Papillomavirus (HPV)—based pseudoviral gene delivery system for the non-viral, Episomally Replicating Vector pEPI-1,” Gene Ther Mol Biol, 9:371-376 (2005)], HBV, and HCMV can also be used.

Methods for specifically targeting a cell type with a viral vector are known in the art. U.S. Patent Publication No. 2007/0020238 describes the delivery of DNA to targeted cells using viral vectors as gene delivery vehicles. For targeting cancer cells, for example, viral delivery systems can be designed to target the particular cell type affected by cancer.

A preferred alternative for delivering restriction endonucleases to cells in need of treatment according to the present methods is to deliver such enzyme proteins directly to cells rather than transfecting cells with nucleic acids which must be translated in order to produce restriction endonucleases. One approach to delivering restriction endonuclease proteins to cells is to bind the nuclease to the capsid or other structural protein of a virus [as set out in Asokan et al., “Bioluminescent virion shells: new tools for quantitation of AAV vector dynamics in cells and live animals,” Gene Therapy, 15:1618-1622 (2008)]. For example, an endonuclease as described herein can be incorporated into recombinant AAV serotypes 1,2 and 8 capsids by fusing the endonuclease to the N-terminus of the VP2 capsid subunit, thereby forming virion shells. When endonuclease proteins are directly delivered to a host cell by a viral vector, it is preferred that no viral polynucleotides be transferred, as in the foregoing method utilizing AAV virion shells.

Another approach allowing the direct transfer of proteins to cells with a viral vector is described in Pepperl-Klindworth et al., “Protein delivery by subviral particles of human cytomegalovirus,” Gene Therapy, 10:278-284 (2003). In this method, proteins are delivered by subviral particles derived from the human cytomegalovirus (HCMV), termed dense bodies (DB), which are non-infectious. The dense bodies comprise fusion proteins made by fusing the carboxiterminus of the tegument protein (pp65) of HCMV to an endonuclease for use in the present methods. The dense bodies include such fusion proteins but lack viral capsids or DNA. Delivery of such dense bodies to fibroblasts and other cell types susceptible to infection by HCMV is possible.

An alternative approach to directly delivering restriction endonuclease proteins to cells is to include peptide sequences, such as arginine-rich sequences from HIV Tat, to the endonuclease in order to promote protein penetration into cells and tissues. Several such protein transduction domain (PTD) transporter peptides have been described, for example in Siprashvili et al., “Intracellular Delivery of Functional Proteins via Decoration with Transporter Peptides,” Molecular Therapy, 9:721-728 (2004).

In an alternative embodiment, polynucleotides encoding restriction endonucleases that can be translated within a host cell are delivered by viral vectors in the present method. A number of viral vectors for delivering polynucleotides can be used. For example, vectors targeting neurons that can be used include those derived from HSV, including amplicon vectors, replication-defective HSV and attenuated HSV (Krisky et al., 1998, Gene Ther. 5: 1517-30). Other vectors that have been developed for gene therapy uses can also be used with the methods of the invention. Such vectors include those derived from baculoviruses and alpha-viruses (Jolly, 1999, The development of human gene therapy, New York: Cold Spring Harbor Lab, 209-40).

A vector containing a polynucleotide encoding the restriction endonuclease of interest is preferably operably linked to a promoter which is used to drive expression of the restriction endonuclease. A promoter contains untranslated sequences that are generally located upstream (5′) from the start codon of a gene (generally within about 100 to 1000 bp) and control the transcription and translation of the polynucleotide sequence to which they are operably linked. Promoters can be inducible or constitutive. Inducible promoters initiate increased levels of transcription from DNA under their control in response to some change in culture conditions, such as a change in temperature. One of skill in the art will be able to select an appropriate promoter based on the specific circumstances.

Transcription can be increased by inserting an enhancer sequence into the vector. Enhancers are typically cis-acting elements of DNA, usually about 10 to 300 bp in length, that act on a promoter to increase its transcription. Many enhancer sequences are now known from mammalian genes (globin, elastase, albumin, alpha-fetoprotein, and insulin) and eukaryotic cell viruses (SV40 enhancer, the cytomegalovirus early promoter enhancer, the polyoma enhancer on the late side of the replication origin, and adenovirus enhancers). The enhancer can be spliced into the vector at a position 5′ or 3′ to the polynucleotide sequence encoding the restriction endonuclease. Expression vectors will also preferably contain sequences necessary for the termination of transcription and for stabilizing the mRNA. These sequences are often found in the 5′ and, occasionally 3′, untranslated regions of eukaryotic or viral DNAs or cDNAs and are well known in the art.

Other methods for transferring restriction endonucleases or polynucleotides encoding such endonucleotides include the use of lipid reagents, as described in Example 2 below, or liposomes. Liposomes are microscopic vesicles that consist of one or more lipid bilayers surrounding aqueous compartments (Bakker-Woudenberg et al., 1993, Eur. J. Clin. Microbiol. Infect. Dis. 12 (Suppl. 1): S61 (1993); Kim, 1993, Drugs 46: 618). Liposomes can be unilamellar or multilamellar, and can vary in size with diameters ranging from 0.02 μm to greater than 10 μm. Liposomes can be prepared for targeting to particular cells or organs by varying their phospholipid composition or by inserting cellular receptors or ligands into the liposomes. Preferably, liposomes are delivered directly to tissues in need of treatment.

### Methods of Treatment

The methods of the present invention treat a medical condition which involves the presence of an undesired polynucleotide in a predetermined cell type. Such medical conditions include cancer and viral infections.

Prior attempts to administer exogenously-derived restriction endonucleases to a subject in order to treat a medical condition have not been successful. With respect to the treatment of RNA virus infections, such as HIV infections, exogenous restriction endonucleases will have no effect on circulating viruses, since restriction endonucleases act on DNA and not RNA. For this reason, the treatments of the present invention contemplate the delivery of restriction endonucleases to cells so that such endonucleases can act on DNA within infected cells, such as proviral DNA.

In the case of a viral infection with, for example, a virus such as HIV, which uses reverse transcriptase to transcribe its RNA genome into DNA followed by integration of the viral DNA into the host DNA, the undesired viral DNA can be excised from the host DNA using restriction endonucleases which recognize and cleave the viral DNA from the host DNA. HIV begins its life cycle when it binds to a CD4 receptor and one of two co-receptors on the surface of a CD4⁺T-lymphocyte. The virus then fuses with the host cell. After fusion, the virus releases RNA, its genetic material, into the host cell. An HIV enzyme called reverse transcriptase then converts the single-stranded HIV RNA to double-stranded HIV DNA. Following this, HIV DNA enters the host cell's nucleus, where an HIV enzyme called integrase integrates the HIV DNA into the host cell's own DNA. The integrated HIV DNA is called a provirus. To actively produce the virus, certain cellular transcription factors need to be present, the most important of which is NF-κB (NF kappa B), which is upregulated when T-cells become activated. When the host cell receives such an activation signal, the provirus uses a host enzyme called RNA polymerase to create copies of the HIV genomic material, as well as shorter strands of messenger RNA (mRNA) which are used as a blueprint to make long chains of HIV proteins. Such proteins are then assembled with copies of HIV's RNA genetic material to produce new virus particles.

Since the HIV virus can integrate into host DNA and exist as a provirus, the only way to eradicate the virus from a cell infected in this way is to remove the proviral DNA from the infected cell. The present method accomplishes this through the administration of a restriction endonuclease to such a cell which is adapted to excise the proviral DNA.

The viral DNA to be targeted includes DNA encoding conserved protein domains essential for viral production. In the case of DNA, reverse transcriptase does not have an editing function like that of human polymerases, and therefore one round of DNA synthesis results in as many as ten mutations in the HIV genome. This accounts for the great genetic and antigenic variation of HIV, and the constant emergence of new HIV strains. However, some regions of the HIV genome that encode protein domains essential for viral replication and virion production are conserved. Such conserved regions are therefore preferably targeted by the restriction endonucleases used in the present method.

Preferably, the endonucleases used in the present methods excise the entire proviral sequence, either by cutting the proviral sequence at its respective ends, or less preferably by cutting a host sequence on either side of the proviral sequence. In an alternative embodiment, less than the entire proviral sequence can be excised, as long as the portion of the proviral sequence that is cut results in the virus being unable to replicate, and preferably also reduces or eliminates harm to the cell caused by viral production.

A wide variety of other viral infections, including infections caused by viruses with genomes of double-stranded DNA during at least part of their life-cycle, can be treated with the restriction endonucleases of the present invention. Notable among the double stranded DNA viruses that infect man and cause health problems are the Hepadnaviridae, for instance, hepatitis B virus; the Papovaviridae, including human papillomaviruses (HPV) 1-48, some of which are associated with the development of carcinomas; the Adenoviridae; and the Herpesviridae, including, for instance, herpes simplex viruses 1 and 2, cytomegalovirus and Epstein-Barr virus. Many of these viruses are characterized by prolonged persistence in the body in a latent form, and recurrent clinical disease which can occur throughout life. Among the viruses whose genomes are double-stranded DNA during only part of their life-cycles are the parvoviruses and the retroviruses. Notable among the retroviruses of the latter class is HIV.

Any type or stage of cancer can also be treated with the restriction endonuclease of the present invention. For example, lung cancer, colon cancer, breast cancer, testicular cancer, stomach cancer, pancreatic cancer, ovarian cancer, liver cancer, bladder cancer, colorectal cancer, prostate cancer, and hematopoitiec cancers such as leukemia can be treated with the restriction endonuclease of the present invention. Following the identification of a nucleotide sequence of the cancer cell that can be cleaved in order to treat the cancer, such as an overexpressed growth factor, an appropriate restriction endonuclease capable of cleaving the nucleotide sequence can be identified. A nucleotide sequence coding for the restriction endonuclease, under the control of an appropriate promoter, is prepared, and this nucleotide sequence is packaged in a vector targeting the particular cancer cell. The vector is then administered to a subject in need of treatment. Alternatively, restriction endonucleases can be directly delivered to cancer cells, such as with a viral vector, as described above.

Intracellular bacteria, such as mycobacterium tuberculosis and mycobacterium leprae can also be treated with the present method. Such bacteria invade and replicate within the cells of a subject. Restriction endonucleases directed against bacterial DNA can be administered to infected cells order to treat such bacteria. In this embodiment, the restriction endonucleases that are delivered are adapted to cut bacterial DNA sequences carried by such bacteria.

In addition, certain genetic disorders can be treated according to the present methods. Preferably, the genetic disorders treated by the present methods are single-gene disorders, caused by one or more mutations occurring in the DNA sequence of one gene. The types of genetic disorders treatable by the present methods are generally those in which a defective protein or other product of a mutant gene causes harm to a subject, such that the cleaving or excision of the mutant gene reduces or eliminates the harmful gene product. For example, in individuals with alpha-1 antitrypsin deficiency, the presence of a mutation in the alpha-1 antitrypsin sequence results in the production of an abnormal, misfolded alpha-1 antitrypsin protein which aggregates and accumulates in liver cells. Retention of the misfolded aggregate leads to the development of cirrhosis. In the present method, the DNA producing such abnormal aggregated proteins can be cleaved in liver cells by the targeted delivery of an appropriate restriction endonuclease (or the DNA coding for such an endonuclease) to liver cells. For example, a hepatitis B virus (HBV)-based vector [as taught in Untergasser, U., “Hepatitis B Virus-Based Vectors Allow the Elimination of Viral Gene Expression and the Insertion of Foreign Promoters,” Human Gene Therapy, 15: 203-210 (2004)] can be used to deliver an appropriate restriction endonuclease to liver cells in order to treat the cirrhosis associated with alpha-1 antitrypsin deficiency. In this embodiment the restriction endonuclease targets the SERPINA1 gene sequence, and preferably only SERPINA1 gene sequences containing a deleterious mutation.

The present methods can similarly treat other genetic or acquired medical conditions involving the accumulation of aberrant proteins in cells, such as Alzheimer's disease, including early-onset (genetic) Alzheimer's disease (amyloid precursor protein and other proteins), Parkinson's disease (torsinA and other proteins), Huntington's disease (TATA-binding protein and other proteins), amyotrophic lateral sclerosis, transmissible spongiform encephalopathies (prion protein), inclusion body myopathy, and the systemic amyloidoses. Transmissible spongiform encephalopathies (prion diseases) include classic Creutzfeldt-Jakob disease, new variant Creutzfeldt-Jakob disease, Gerstmann-Sträussler-Scheinker syndrome, and fatal familial insomnia. Such treatment is effected by delivering restriction endonucleases (or polynucleotides encoding such restriction endonucleases) to the affected cells and cutting the DNA that encodes such aberrant proteins. When the affected cells are neurons, as in Alzheimer's disease and Huntington's disease, a viral vector such as an HSV vector can be use, for example.

The present methods can also be used to treat genetic disorders associated with genetic mutations when the protein product or function of the mutated gene is not known. For example, Huntington's disease is associated with mutations in the prion protein gene (HDL1), the junctophilin 3 gene (HDL2), a recessively inherited HTT gene (HDL3), and the gene encoding the TATA box-binding protein (HDL4/SCA17). Preferably, only genes with mutated sequences are targeted by the restriction endonucleases delivered according to the present methods.

Typically, for therapeutic applications, one or more restriction endonucleases or a vector encoding one or more restriction endonucleases can be combined with a pharmaceutically acceptable excipient appropriate to a planned route of administration. A variety of pharmaceutically acceptable excipients are well known, from which those that are effective for delivering the restriction endonuclease to a specific site can be selected. The Handbook of Pharmaceutical Excipients published by the American Pharmaceutical Association is one useful guide to appropriate excipients for use in the invention. A composition is said to be a “pharmaceutically acceptable excipient” if its administration can be tolerated by the recipient. Sterile phosphate-buffered saline is one example of a pharmaceutically acceptable excipient that is appropriate for intravenous administration.

For purposes of treatment, one or more restriction endonucleases or a vector encoding one or more restriction endonucleases, and a pharmaceutically acceptable excipient are administered in a therapeutically effective amount. Such a combination is said to be administered in a “therapeutically effective amount” if the amount administered is physiologically significant, i.e. if its presence results in a detectable change in the physiology of the recipient. In the present context, an agent is physiologically significant if its presence results in a decrease in the severity of one or more symptoms of a medical condition caused by undesired DNA in the subject.

Administration can be topical or internal, or by any other suitable avenue for introducing a therapeutic agent to a patient. Topical administration can be by application to the skin, or to the eyes, ears, or nose. Internal administration can proceed intradermally, subcutaneously, intramuscularly, intraperitoneally, intraarterially or intravenously, or by any other suitable route. It also may in some cases be advantageous to administer a composition of the invention by oral ingestion, by respiration, rectally, or vaginally. For a brief review of pharmaceutical dosage forms and their use, see Pharmaceutical Dosage Forms and Their Use (1985, Hans Huber Publishers, Berne, Switzerland).

### EXAMPLES

### Example 1

### Delivery of Restriction Endonucleases: AAV Vector

The AAV capsid is comprised of three viral protein subunits, Vp1, Vp2 and Vp3. The N-terminus of Vp2 has been found to tolerate fusion with large proteins such as GFP (˜30 kDa), thereby allowing incorporation and surface display of non-endogenous viral proteins on the AAV onto the N-terminus of the Vp2 protein derived from the AAV2 capsid sequence.

To create a Vp2 protein displaying a restriction endonuclease, the polynucleotide sequence of such an endonuclease is first inserted into a plasmid such as pVp2AGFP (in which the GFP sequence has preferably first been removed). This plasmid is then co-transfected into HEK 293 cells with another plasmid coding for the Vp1 and Vp3 subunits, such as the pXR-ACA plasmid [the foregoing plasmids are described in Grieger et al., “Production and characterization of adeno-associated viral vectors,” Nat. Protoc. 1:1412-1428 (2006)]. Helper Ad genes (in plasmid pXX6-80) and packaging construct (in plasmid pTR-GFP) are also transfected [see, Summerford et al., “Membrane-associated heparan sulfate proteoglycan is a receptor for adeno-associated virus type 2 virions,” J. Virol., 72:1438-1445 (1998)]. Virion shells containing the modified Vp2 protein as well as the Vp1 and Vp3 proteins are purified by cesium chloride density ultracentrifugation and then dialyzed against PBS in order to purify them.

### Example 2

### Delivery of Restriction Endonucleases: Lipid Reagent

A restriction endonuclease to be delivered to a subject is suspended in 10 mM HEPES (pH 7.0) and 150 mM NaCl and then mixed with BioPorter lipid reagent, which contains a trifluoroacetylated lipopolyamine mixed with dioleoyl phosphatidylethanolamine (available from Gene Therapy Systems, Inc., San Diego, Calif.). Aliquots of 0.25 μg of endonuclease protein in 10-μl buffer are mixed with 1 μl of dried BioPorter lipids, and the mixture is incubated for 5 min at room temperature. The mixture is diluted in 100 μl of serum-free medium. Aliquots of the prepared mixture are administered to a tissue of a subject in need of treatment until a therapeutic effect is observed.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure. All references cited herein are incorporated by reference in their entirety.

What is claimed is:

1. A method of treating a medical condition of a subject, wherein the medical condition is characterized by the presence of an undesired DNA sequence in a predetermined cell type, comprising the steps of:
(a) providing a viral vector comprising a restriction endonuclease or one or more polynucleotides coding for the restriction endonuclease; and
(b) administering to the subject an amount of the vector capable of treating the medical condition, wherein the restriction endonuclease specifically cleaves the undesired DNA sequence.

2. The method of claim 1, wherein the undesired DNA sequence is present in or derived from a bacterium or a virus.

3. The method of claim 2, wherein the undesired DNA sequence is an HIV proviral DNA sequence.

4. The method of claim 1, wherein the viral vector is a replication defective viral vector.

5. The method of claim 1, wherein the viral vector is derived from a virus selected from the group consisting of AAV, HSV, HIV, HBV, HPV, HCMV, a baculovirus, and an alpha-virus.

6. The method of claim 1, wherein the viral vector comprises a polynucleotides coding for the restriction endonuclease and the polynucleotide is operably linked to a promoter.

7. The method of claim 1, wherein the viral vector comprises a restriction endonuclease.

8. The method of claim 7, wherein the restriction endonuclease is fused to a viral protein.

9. The method of claim 8, wherein the viral protein is derived from HCMV or AAV.

10. The method of claim 7, wherein the restriction endonuclease further comprises a protein transduction domain transporter peptide.

11. The method of claim 1, wherein the medical condition is selected from the group consisting of lung cancer, colon cancer, breast cancer, testicular cancer, stomach cancer, pancreatic cancer, ovarian cancer, liver cancer, bladder cancer, colorectal cancer, leukemia, and prostate cancer.

12. The method of claim 1, wherein the medical condition is a genetic disorder.

13. The method of claim 13, wherein the genetic disorder is selected from the group consisting of alpha-1 antitrypsin deficiency, Huntington's disease, and early-onset Alzheimer's disease.

14. The method of claim 1, wherein the medical condition is a viral infection caused by a virus from a family selected from the group consisting of Hepadnaviridae, Papovaviridae, Adenoviridae, and Herpesviridae.

15. The method of claim 14, wherein the medical condition is a viral infection caused by a virus selected from the group consisting of hepatitis B virus, human papillomavirus, herpes simplex virus 1, herpes simplex virus 2, cytomegalovirus, and Epstein-Barr virus.

16. The method of claim 2, wherein the bacterium is an intracellular bacterium selected from the group consisting of mycobacterium tuberculosis and mycobacterium leprae.

17. The method of claim 1, wherein the medical condition is cancer.

18. The method of claim 1, wherein the medical condition is selected from the group consisting of Parkinson's disease, a transmissible spongiform encephalopathy, and amyotrophic lateral sclerosis.

19. The method of claim 1, further comprising the step of combining the vector with a pharmaceutically acceptable excipient prior to administration.
